(19) United States
(12) Patent Application Publication
(10) Pub. No.: US 2010/0233096 A1
(43) Pub. Date: Sep. 16, 2010

(54) METHOD TO PRODUCE HYPERPOLARISED CARBOXYLATES AND SULPHONATES

(76) Inventors: Mathilde H. Lerche, Frederiksberg C (DK); Magnus Karlsson, Malmo (SE)

Correspondence Address: GE HEALTHCARE, INC., IP DEPARTMENT 101 CARNEGIE CENTER, PRINCETON, NJ, 08540-6231, US

(21) Appl. No.: 12/294,937

(22) PCT Filed: Mar. 21, 2007
(86) PCT No.: PCT/NO2007/000109

§ 371 Date: Sep. 29, 2008

(30) Foreign Application Priority Data
Mar. 29, 2006 (NO) 20061435

(51) Int. Cl. A61K 49/10

(52) U.S. Cl. 424/9.36; 424/9.3

(57) ABSTRACT

The invention relates to a dynamic nuclear polarisation method for producing hyperpolarised carboxylates or sulphonates or mixtures thereof wherein the carboxylate or sulphonate used in the method of the invention comprises certain inorganic cations. The invention further relates to compositions for use in that method.

The invention relates to a dynamic nuclear polarisation method for producing hyperpolarised carboxylates or sulphonates or mixtures thereof wherein the carboxylate or sulphonate used in the method of the invention comprises certain inorganic cations. The invention further relates to compositions for use in that method.

Magnetic resonance (MR) imaging (MRI) is an imaging technique that has become particularly attractive to physicians as it allows for obtaining images of a patient's body or parts thereof in a non-invasive way and without exposing the patient and the medical personnel to potentially harmful radiation such as X-ray. Because of its high quality images, MRI is the favoured imaging technique of soft tissue and organs and it allows for the discrimination between normal and diseased tissue, for instance tumours and lesions.

MRI may be carried out with or without MR contrast agents. However, contrast-enhanced MRI usually enables the detection of much smaller tissue changes which makes it a powerful tool for the detection of early stage tissue changes like for instance small tumours or metastases.

Several types of contrast agents have been used in MRI. Water-soluble paramagnetic metal chelates, for instance gadolinium chelates like Omniscan™ (GE Healthcare) are widely used MR contrast agents. Because of their low molecular weight they rapidly distribute into the extracellular space (i.e. the blood and the interstitium) when administered into the vasculature. They are also cleared relatively rapidly from the body.

Blood pool MR contrast agents on the other hand, for instance superparamagnetic iron oxide particles, are retained within the vasculature for a prolonged time. They have proven to be extremely useful to enhance contrast in the liver but also to detect capillary permeability abnormalities, e.g. “leaky” capillary walls in tumours which are a result of tumour angiogenesis.

Despite the undisputed excellent properties of the aforementioned contrast agents their use is not without any risks. Although paramagnetic metal chelates have usually high stability constants, it is possible that toxic metal ions are released in the body after administration. Further, these type of contrast agents show poor specificity.

WO-A-99/35508 discloses a method of MR investigation of a patient using a hyperpolarised solution of a high T₁ agent as MRI contrast agent. The term “hyperpolarisation” means enhancing the nuclear polarisation of NMR active nuclei present in the high T₁ agent, i.e. nuclei with non-zero nuclear spin, preferably ¹³C- or ¹⁵N-nuclei. Upon enhancing the nuclear polarisation of NMR active nuclei, the population difference between excited and ground nuclear spin states of these nuclei is significantly increased and thereby the MR signal intensity is amplified by a factor of hundred and more. When using a hyperpolarised ¹³C- and/or ¹⁵N-enriched high T₁ agent, there will be essentially no interference from background signals as the natural abundance of ¹³C and/or ¹⁵N is negligible and thus the image contrast will be advantageously high. The main difference between conventional MRI contrast agents and these hyperpolarised high T₁ agents is that in the former changes in contrast are caused by affecting the relaxation times of water protons in the body whereas the latter class of agents can be regarded as non-radioactive tracers, as the signal obtained arises solely from the agent.

A variety of possible high T₁ agents for use as MR imaging agents are disclosed in WO-A-99/35508, including non-endogenous and endogenous compounds like acetate, pyruvate, oxalate or gluconate, sugars like glucose or fructose, urea, amides, amino acids like glutamate, glycine, cysteine or aspartate, nucleotides, vitamins like ascorbic acid, penicillin derivates and sulphonamides. It is further stated that intermediates in metabolic cycles such as the citric acid cycle like fumaric acid and pyruvic acid are preferred imaging agents for MR imaging of metabolic activity.

Hyperpolarised MR imaging agents that play a role in the metabolic processes in the human and non-human animal body are of great interest, as these hyperpolarised imaging agents can be used to get information about the metabolic state of a tissue in an in vivo MR investigation, i.e. they are useful for in vivo imaging of metabolic activity. Information of the metabolic status of a tissue might for instance be used to discriminate between healthy and diseased tissue.

Pyruvate is a compound that plays a role in the citric acid cycle and the conversion of hyperpolarised ¹³C-pyruvate to its metabolites hyperpolarised ¹³C-lactate, hyperpolarised ¹³C-bicarbonate and hyperpolarised ¹³C-alanine can be used for in vivo MR studying of metabolic processes in the human body. Hyperpolarised ¹³C-pyruvate may for instance be used as an MR imaging agent for in vivo tumour imaging as described in detail in WO-A-2006/011810 and for assessing the viability of myocardial tissue by MR imaging as described in detail in WO-A-2006/054903.

It has to be stressed that the signal of a hyperpolarised imaging agent decays due to relaxation and—upon administration to the patient's body—dilution. Hence the T₁ value of the imaging agents in biological fluids, e.g. blood must be sufficiently long (or high in terms of WO-A-99/35508) to enable the agent to be distributed to the target site in the patient's body in a highly hyperpolarised state. Apart from the imaging agent having a long T₁, it is extremely important and favourable to achieve a high polarisation level.

Several hyperpolarising techniques are disclosed in WO-A-99/35508, one of them is the dynamic nuclear polarisation (DNP) technique whereby polarisation of MR active nuclei in a compound to be polarised, i.e. a sample, is effected by a polarisation agent or so-called DNP agent, a compound comprising unpaired electrons. During the DNP process, energy, normally in the form of microwave radiation, is provided, which will initially excite the DNP agent. Upon decay to the ground state, there is a transfer of polarisation from the unpaired electron of the DNP agent to the NMR active nuclei of the sample. Generally, a moderate or high magnetic field and a very low temperature are used in the DNP process, e.g. by carrying out the DNP process in liquid helium and a magnetic field of about 1 T or above. Alternatively, a moderate magnetic field and any temperature at which sufficient polarisation enhancement is achieved may be employed. The DNP technique is for example described in WO-A-98/58272 and in WO-A-01/96895, both of which are included by reference herein.

The DNP agent plays a decisive role in the DNP process as its choice has a major impact on the level of polarisation that can be achieved. A variety of DNP agents—in WO-A-99/35508 denoted “OMRI contrast agents”—is known. The use of oxygen-based, sulphur-based or carbon-based stable trityl radicals as described in WO-A-99/35508, WO-A-88/10419, WO-A-90/00904, WO-A-91/12024, WO-A-93/02711, WO-A-98/39277 and WO-A-96/39367 as DNP agents has resulted in high levels of polarisation in a variety of different samples.

It has also been found that for the transfer of polarisation from the DNP agent to the NMR active nuclei of the sample during the DNP process it is necessary that DNP agent and sample are in intimate contact. This intimate contact can be achieved by choosing a DNP agent that is soluble in the sample. Further, it is important to have a homogeneous distribution of the DNP agent in the sample. For samples which crystallize upon cooling/freezing, low polarisation levels or even no polarisation has been obtained. However, it has been reported earlier that polarisation levels in samples which crystallize upon cooling/freezing can be improved by adding glass formers, since a mixture of a DNP agent, a sample and a glass former forms an amorphous solid (“glass”) upon cooling/freezing (Ardenkjær-Larsen et al., PNAS 100(18), 10158-10163, 2003).

Suitable glass formers are for instance glycerol, propanediol or glycol. However, the addition of glass formers has usually to be kept to the necessary minimum as this addition “dilutes” the sample which is a disadvantage for certain applications like the use of the hyperpolarised sample as an imaging agent in MRI. In this case the hyperpolarised sample needs to be administered to the patient at a high concentration, i.e. a highly concentrated sample must be used in the DNP process. In this context, it is also important that the mass of the frozen composition containing the sample (i.e. DNP agent, sample and if necessary glass formers and/or solvents) is kept as small as possible as a high mass will have a negative impact on the efficiency of the dissolution process, if dissolution is used to transfer the solid hyperpolarised composition after the DNP process into the liquid state, e.g. for using it as an imaging agent. This is due to the fact that for a given volume of solvent in the dissolution process, the sample mass to solvent ratio decreases when the sample mass is increased. Further, if the polarised sample is intended to be used as an imaging agent, the added glass formers may need to be removed before the imaging agent is administered into a patient.

A considerably large number of metabolically active compounds are carboxylates, i.e. salts of carboxylic acids. Examples are pyruvate, lactate, bicarbonate, succinate, malate, fumarate, citrate, isocitrate, a-ketoglutarate or oxaloacetate. These compounds are commercially available in form of their sodium salts and most of them can be dissolved in water and mixed with a DNP agent to prepare a composition for the DNP process. However, upon cooling/freezing, these mixtures may crystallize which—without the addition of glass formers—leads to polarisation levels which are too low to use the polarised carboxylates as MR imaging agents for MR imaging of metabolic activity. Some of the aforementioned compounds like pyruvate and lactate may be polarised in form of their free acids since these acids are liquids at room temperature which makes it possible to directly dissolve the DNP agent in these liquids. The liquid acid/DNP mixture does not crystallize upon cooling/freezing and hence the addition of glass formers is not necessary. The disadvantage is that the DNP agent has to be stable in these acids, a criterion which considerably narrows the range of suitable DNP agents. Further, during the dissolution step or afterwards, a base has to be used to convert the free acid into the carboxylate. This also requires consumables (vessels, bottles, tubing etc.) that can withstand strong acids and bases.

We have now found a method to polarise carboxylates without the addition of glass formers. It has been found that a solution of a carboxylate comprising an inorganic cation from the group consisting of NH₄⁺, K⁺, Rb⁺, Cs⁺, Ca²⁺, Sr²⁺ and Ba²⁺ can be polarised by dynamic nuclear polarisation without the addition of glass formers since such a solution does not crystallize upon cooling/freezing. The advantage is that the “dilution” of the polarised carboxylate by any glass formers and the removal of the glass formers from the polarised carboxylate is no longer an issue. Thus, for a given size of a sample cup to hold a sample to be polarised by DNP, a much higher concentration of carboxylate can be polarised. A further advantage of the direct polarisation of carboxylates is that the indirect route of polarising the free carboxylic acid and all the disadvantages of this route as outlined in the paragraph above can be avoided. This results in the possibility to use a broader range of DNP agents as these agents no longer have to be stable in the acids to be polarised. It has further been found that the method as described above also may be used for the DNP polarisation of sulphonates.

Thus viewed form one aspect the invention provides a method of producing a hyperpolarised carboxylate or sulphonate or mixtures thereof, the method comprising
a) preparing a solution comprising a carboxylate or a sulphonate or mixtures thereof wherein the carboxylate and/or sulphonate comprises an inorganic cation from the group consisting of NH₄⁺, K⁺, Rb⁺, Cs⁺, Ca²⁺, Sr²⁺ and Ba²⁺, a DNP agent and optionally a paramagnetic metal ion;
b) freezing the solution;
c) carrying out dynamic nuclear polarisation on the frozen solution to obtain a frozen solution comprising the hyperpolarised carboxylate or the hyperpolarised sulphonates or mixtures thereof; and
d) optionally liquefying the frozen solution obtained in step c).

The terms “hyperpolarised” and “polarised” are used interchangeably hereinafter and denote a nuclear polarisation level in excess of 0.1%, more preferred in excess of 1% and most preferred in excess of 10%.

The level of polarisation may for instance be determined by solid state NMR measurements of the NMR active nucleus in the frozen hyperpolarised sample. For instance, if the NMR active nucleus in the hyperpolarised sample is ¹³C, a solid state ¹³C-NMR of said sample is acquired. The solid state ¹³C-NMR measurement preferably consists of a simple pulse-acquire NMR sequence using a low flip angle. The signal intensity of the hyperpolarised sample in the NMR spectrum is compared with signal intensity of the sample in a NMR spectrum acquired before the dynamic nuclear polarisation process. The level of polarisation is then calculated from the ratio of the signal intensities of before and after DNP.

In a similar way, the level of polarisation for dissolved hyperpolarised samples may be determined by liquid state NMR measurements of the NMR active nucleus in the liquid hyperpolarised sample. Again the signal intensity of the dissolved hyperpolarised sample is compared with the signal intensity of the dissolved sample before the dynamic nuclear polarisation process. The level of polarisation is then calculated from the ratio of the signal intensities of sample before and after DNP.

The terms “a carboxylate” denotes a salt of a carboxylic acid and the term “a sulphonate” denotes a salt of sulphonic acid. A salt is an ionic compound composed of cations and anions. In the method of the invention, said cations are inorganic cations from the group consisting of NH₄⁺, K⁺, Rb⁺, Cs⁺, Ca²⁺, Sr²⁺ and Ba²⁺ and said anions are carboxylate anions or sulphonates anions. In the following the terms “carboxylate” and “sulphonates” denote a salt of a carboxylic acid/sulphonic acid or a carboxylic acid anion/sulphonic acid anion. It is apparent from the context when said terms denote the salt or the anion of the salt.

The term “carboxylate/sulphonate” used in the following paragraphs means that the statements made in these paragraphs equally apply to carboxylates and sulphonates.

Although written in the singular form the terms “a carboxylate” and “a sulphonates” denote a chemical entity or entities, e.g. a certain carboxylate or a certain sulphonate but also several different carboxylates or several different sulphonates, i.e. mixtures of several different carboxylates or mixtures of several different sulphonates. This is illustrated in the following paragraph with carboxylates, but applies likewise to sulphonates.

As an example pyruvate is a certain carboxylate and the method of the invention can be used to produce hyperpolarised pyruvate by preparing in step a) a solution comprising a pyruvate that comprises an inorganic cation from the group consisting of NH₄⁺, K⁺, Rb⁺, Cs⁺, Ca²⁺, Sr²⁺ and Ba²⁺, a DNP agent and optionally a paramagnetic metal ion. Concrete examples of such solutions are for instance a solution comprising Cs-pyruvate, a DNP agent and optionally a paramagnetic metal ion or a solution comprising Sr-pyruvate, a DNP agent and optionally a paramagnetic metal ion. Another example of a certain carboxylate is bicarbonate and the method of the invention can be used to produce hyperpolarised bicarbonate by preparing in step a) a solution comprising a bicarbonate that comprises an inorganic cation from the group consisting of NH₄⁺, K⁺, Rb⁺, Cs⁺, Ca²⁺, Sr²⁺ and Ba²⁺, a DNP agent and optionally a paramagnetic metal ion. Concrete examples of such solutions are for instance a solution comprising Cs-bicarbonate, a DNP agent and optionally a paramagnetic metal ion or a solution comprising Rb-bicarbonate, a DNP agent and optionally a paramagnetic metal ion.

Further, as an example pyruvate and lactate are several different carboxylates and the method of the invention can be used to produce a mixture of hyperpolarised pyruvate and hyperpolarised lactate by preparing in step a) a solution comprising a pyruvate that comprises an inorganic cation from the group consisting of NH₄⁺, K⁺, Rb⁺, Cs⁺, Ca²⁺, Sr²⁺ and Ba²⁺ and a lactate that comprises an inorganic cation from the group consisting of NH₄⁺, K⁺, Rb⁺, Cs⁺, Ca²⁺, Sr²⁺ and Ba²⁺, a DNP agent and optionally a paramagnetic metal ion. Concrete examples of such solutions are for instance a solution comprising Cs-pyruvate, Rb-lactate, a DNP agent and optionally a paramagnetic metal ion or a solution comprising Cs-pyruvate, Cs-lactate, a DNP agent and optionally a paramagnetic metal ion.

In line with the definitions provided above, the term “or mixtures thereof” denotes i) a mixture of a certain carboxylate and a certain sulphonate or ii) a mixture of several different carboxylates and a certain sulphonate or iii) a mixture of a certain carboxylate and several different sulphonates or iv) a mixture of several different carboxylates and several different sulphonates. This is illustrated in the following paragraph.

As an example for i) a solution is prepared in step a) of the method of the invention wherein said solution comprises a pyruvate comprising an inorganic cation from the group consisting of NH₄⁺, K⁺, Rb⁺, Cs⁺, Ca²⁺, Sr²⁺ and Ba²⁺, a methanesulphonate comprising an inorganic cation from the group consisting of NH₄⁺, K⁺, Rb⁺, Cs⁺, Ca²⁺, Sr²⁺ and Ba²⁺, a DNP agent and optionally a paramagnetic metal ion. As an example for ii) a solution is prepared in step a) of the method of the invention wherein said solution comprises a pyruvate comprising an inorganic cation from the group consisting of NH₄⁺, K⁺, Rb⁺, Cs⁺, Ca²⁺, Sr²⁺ and Ba²⁺, a bicarbonate comprising an inorganic cation from the group consisting of NH₄⁺, K⁺, Rb⁺, Cs⁺, Ca²⁺, Sr²⁺ and Ba²⁺, a methanesulphonate comprising an inorganic cation from the group consisting of NH₄⁺, K⁺, Rb⁺, Cs⁺, Ca²⁺, Sr²⁺ or Ba²⁺, a DNP agent and optionally a paramagnetic metal ion. As an example for iii) a solution is prepared in step a) of the method of the invention wherein said solution comprises a pyruvate comprising an inorganic cation from the group consisting of NH₄₊, K⁺, Rb⁺, Cs⁺, Ca²⁺, Sr²⁺ and Ba²⁺, a methanesulphonate comprising an inorganic cation from the group consisting of NH₄₊, K⁺, Rb⁺, Cs⁺, Ca²⁺, Sr²⁺ and Ba²⁺, a benzenesulphonate comprising an inorganic cation from the group consisting of NH₄₊, K⁺, Rb⁺, Cs⁺, Ca²⁺, Sr²⁺ and Ba²⁺, a DNP agent and optionally a paramagnetic metal ion. As an example for iv) a solution is prepared in step a) of the method of the invention wherein said solution comprises a pyruvate comprising an inorganic cation from the group consisting of NH₄⁺, K⁺, Rb⁺, Cs⁺, Ca²⁺, Sr²⁺ and Ba²⁺, a bicarbonate comprising an inorganic cation from the group consisting of NH₄⁺, K⁺, Rb⁺, Cs⁺, Ca²⁺, Sr²⁺ and Ba²⁺, a methanesulphonate comprising an inorganic cation from the group consisting of NH₄⁺, K⁺, Rb⁺, Cs⁺, Ca²⁺, Sr²⁺ and Ba²⁺, a benzenesulphonate comprising an inorganic cation from the group consisting of NH₄⁺, K⁺, Rb⁺, Cs⁺, Ca²⁺, Sr²⁺ and Ba²⁺, a DNP agent and optionally a paramagnetic metal ion.

In a preferred embodiment, the solution prepared in step a) of the method of the invention comprises a carboxylate, i.e. a certain carboxylate or several different carboxylates which comprise an inorganic cation from the group consisting of NH₄⁺, K⁺, Rb⁺, Cs⁺, Ca²⁺, Sr²⁺ and Ba²⁺.

Preferred inorganic cations are NH₄⁺, K⁺, Rb⁺, Cs⁺, more preferred inorganic cations are K⁺, Rb⁺ and Cs⁺ and most the most preferred inorganic cations are Rb⁺ and Cs⁺.

The carboxylate in the context of the present invention may be the salt of a monocarboxylic acid like for instance carbonic acid, acetic acid, palmitic acid, oleic acid, pyruvic acid or lactic acid. In another embodiment, the carboxylate may be the salt of a di- or polycarboxylic acid like for instance malic acid, fumaric acid, succinic acid, malonic acid, or citric acid. In case of the carboxylate being the salt of a di- or polycarboxylic acid, the salt may be a monocarboxylate, dicarboxylate or a polycarboxylate. For instance in case of citric acid, a tricarboxylic acid, the carboxylate may be a (mono)citrate, i.e. having 2 free carboxylic groups, a dicitrate, i.e. having 1 free carboxylic group or a tricitrate, i.e. having no free carboxylic groups. If the carboxylate used in the method of the invention is a carboxylate of a di- or polycarboxylic acid, it is preferred that the carboxylate does not have any free carboxylic groups. As apparent from the examples given above, the carboxylate may be the salt of a saturated carboxylic acid, like for instance acetic acid, of an unsaturated carboxylic acid, like for instance palmitic acid, or of a carboxylic acid comprising other functional groups like hydroxy group, for instance in lactic acid, or carbonyl groups, like for instance in pyruvic acid or amino groups, like for instance in γ-carboxyglutamic acid. With regard to the presence of amino groups, amino acids, i.e. α-amino carboxylic acids are less preferred in the method of the invention since they tend to form internal salts. However, certain amino acids like aspartate and glutamate are suitable amino acids to be used in the method of the invention. Further, the carboxylate may comprise heteroatoms, an example is for instance pyridine-2,3-dicarboxylic acid (quinolinic acid) which contains 2 nitrogen atoms.

Examples of sulphonates are salts of methanesulphonic acid or p-toluolsulphonic acid Again the sulphonate may be a salt of a sulphonic acid comprising other functional groups like hydroxy groups.

Preferred carboxylates/sulphonates are drug candidates, preferably small molecules, e.g. less than 200 Da, and the hyperpolarised drug candidate(s) may be used in NMR assays to for instance determine binding affinity to a certain receptor or in enzyme assays. Such assays are described in WO-A-2003/089656 or WO-A-2004/051300 and they are preferably based on the use of liquid state NMR spectroscopy which means that the solid hyperpolarised drug candidate(s) has to be liquefied after polarisation, preferably by dissolving or melting it. The carboxylate/sulphonate may or may not be isotopically enriched.

In another preferred embodiment, the carboxylate/sulphonate is a compound that is usable as an imaging agent and the hyperpolarised carboxylate/sulphonate is intended to be used as imaging agent in MR imaging and/or chemical shift imaging in living human or non-human animal beings. In this embodiment, preferred carboxylates/sulphonates are endogenous carboxylates/sulphonates, with endogenous carboxylates being the preferred compounds. For imaging of metabolic processes endogenous carboxylates that play a role in a metabolic process in the human or non-human animal body are preferred. Preferred carboxylates are malate, acetate, fumarate, lactate, citrate, pyruvate, bicarbonate, malonate, carbonate, succinate, oxaloacetate, α-ketoglutarate, 2-oxobutanoate, 2-oxo-5-methylpentanoate, γ-carboxyglutamate, pyridine-2,3-dicarboxylate and isocitrate. Most preferred carboxylates are bicarbonate, fumarate, carbonate, acetate, lactate, 2-oxobutanoate, 2-oxo-5-methylpentanoate, γ-carboxyglutamate, pyridine-2,3-dicarboxylate and pyruvate.

If endogenous carboxylates that play a role in a metabolic process in the human or non-human animal body are used as a compound to be polarised in the method of the invention, the hyperpolarised carboxylates obtained by the inventive method are preferably used as imaging agents for in vivo molecular MR imaging and/or chemical shift imaging of metabolic activity in the living human or non-human animal body. Of these carboxylates, those are preferred which contain polarised NMR active nuclei that exhibit slow longitudinal relaxation (T₁) so that polarisation is maintained for a sufficient length of time for transfer into a human or non-human animal body and subsequent imaging. Preferred carboxylates contain NMR active nuclei with longitudinal relaxation time constants (T₁) that are greater than 10 seconds, preferably greater than 30 seconds and even more preferably greater that 60 seconds at a magnetic field strength of 0.01 to 5 T and a temperature in the range of from 20 to 60° C.

Generally, a carboxylate intended to be used as an imaging agent for in vivo MR imaging and/or chemical shift imaging is preferably an isotopically enriched carboxylate, the isotopic enrichment being more preferably an isotopic enrichment of NMR active nuclei, preferably ¹³C and/or ¹⁵N, if at least a nitrogen atom is present. The isotopic enrichment may include either selective enrichments of one or more sites within the carboxylate or uniform enrichment of all sites. Enrichment can for instance be achieved by chemical synthesis or biological labelling, both methods are known in the art and appropriate methods may be chosen depending on the specific carboxylate to be isotopically enriched.

A preferred embodiment of a carboxylate that is intended to be used as an imaging agent in MR imaging/chemical shift imaging is a carboxylate that is isotopically enriched in only one position of the molecule, preferably with an enrichment of at least 10%, more suitably at least 25%, more preferably at least 75% and most preferably at least 90%. Ideally, the enrichment is 100%.

The optimal position for isotopic enrichment is dependent on the relaxation time of the NMR active nuclei. Preferably, carboxylates are isotopically enriched in positions with long Tᵢ relaxation time. ¹³C-enriched carboxylates that are enriched at a carboxyl-C-atom, a carbonyl-C-atom or a quaternary C-atom are preferably used.

Especially preferred carboxylates for use as imaging agents for MR imaging/chemical shift imaging are ¹³C-pyruvate, ¹³C-acetate, ¹³C-lactate, ¹³C-bicarbonate, ¹³C-carbonate and ¹³C-fumarate with ¹³C-pyruvate being most preferred. ¹³C-pyruvate may be isotopically enriched at the C1-position (¹³C₁-pyruvate), at the C2-position (¹³C₂-pyruvate), at the C3-position (¹³C₃-pyruvate), at the C1- and the C2-position (¹³C_{1,2}-pyruvate), at the C1- and the C3-position (¹³C_{1,3}-pyruvate), at the C2- and the C3-position (¹³C_{2,3}-pyruvate) or at the C1-, C2- and C3-position (¹³C_{1,2,3}-pyruvate). The C1-position is the preferred one for the ¹³C isotopic enrichment.

As mentioned above, also sulphonates may be used as MR imaging agents in living human or non-human animal beings. Such sulphonates are preferably isotopically enriched, the isotopic enrichment being more preferably an isotopic enrichment of NMR active nuclei, preferably ¹³C. The isotopic enrichment may include either selective enrichments of one or more sites within the sulphonate or uniform enrichment of all sites. Enrichment can for instance be achieved by chemical synthesis or biological labelling, both methods are known in the art and appropriate methods may be chosen depending on the specific sulphonate to be isotopically enriched.

A preferred embodiment of a sulphonate that is intended to be used as an imaging agent in MR imaging/chemical shift imaging is a sulphonate that is isotopically enriched in only one position of the molecule, preferably with an enrichment of at least 10%, more suitably at least 25%, more preferably at least 75% and most preferably at least 90%. Ideally, the enrichment is 100%.

The optimal position for isotopic enrichment in a sulphonate is dependent on the relaxation time of the NMR active nuclei. Preferably, sulphonates are isotopically enriched in positions with long Tᵢ relaxation time. ¹³C-enriched sulphonates are preferred and of those sulphonates are preferably used that are enriched at a carboxyl-C-atom, at a carbonyl-C-atom, or at a quaternary C-atom, provided of course that these groups are present in the molecule.

For hyperpolarised carboxylates or sulphonates being used as MR imaging agents in living human or non-human animal beings it is preferred to choose an inorganic cation which is physiologically tolerable. Cations which are used in MR imaging agents and which are known to be physiologically very well tolerable are for instance Na⁺ or meglumine and any of the inorganic cations used in the method of the invention might be exchanged by such physiologically very well tolerable cations by methods known in the art like the use of a cation exchange column.

In another preferred embodiment, the hyperpolarised carboxylate/sulphonate obtained by the method of the invention is used in solid state NMR spectroscopy, i.e. optional step d) is not carried out. In solid state NMR spectroscopy the hyperpolarised solid carboxylate/sulphonate may be analysed by either static or magic angle spinning solid state NMR spectroscopy. For solid state NMR the carboxylate/sulphonate is not limited to carboxylates/sulphonates with certain properties or chemical structures and carboxylate anions/sulphonate anions of any size and type can be used as carboxylates/sulphonates in the method of the invention.

Many of the carboxylates/sulphonates to be used in the method of the invention are commercially available compounds. To obtain a specific carboxylate/sulphonate, a commercially available carboxylate/sulphonate may be used as a starting material and the cation contained in the commercially available compound may be exchanged by NH₄⁺, K⁺, Rb⁺, Cs⁺, Ca²⁺, Sr²⁺ or Ba²⁺ using methods known in the art, for instance by using an ion exchange column or cartridge. Briefly, to prepare the desired carboxylate/sulphonate, in a first step an ion exchange column is prepared by charging a suitable chromatography column with the desired inorganic cation of the group consisting of NH₄⁺, K⁺, Rb⁺, Cs⁺, Ca²⁺, Sr²⁺ and Ba²⁺. In a second step the commercially available carboxylate/sulphonate is dissolved in a suitable solvent and the solution obtained is run through the ion exchange column. The eluate is collected and the solvent is preferably removed by methods known in the art as for instance by evaporation or freeze drying to obtain a carboxylate/sulphonate comprising an inorganic cation from the group consisting of NH₄⁺, K⁺, Rb⁺, Cs⁺, Ca²⁺, Sr²⁺ and Ba²⁺ to be used in the method of the invention.

The solution prepared in step a) of the method of the invention is preferably an aqueous solution, especially if the carboxylate/sulphonates is intended to be used as an imaging agent for in vivo MR imaging and/or chemical shift imaging. In another embodiment, the solution is a non aqueous solution. Suitable solvents or solvent mixtures for such non aqueous solutions are or comprise for instance DMSO or methanol. In yet another embodiment the solution comprises a mixture of a solvent and water, like for instance a mixture of DMSO and/or methanol and water.

For hyperpolarised carboxylates/sulphonates intended to be used as imaging agents for in vivo MR imaging and/or chemical shift imaging it is especially important to obtain the hyperpolarised carboxylate/sulphonate in a high concentration, i.e. by preparing a concentrated solution to be used in the DNP process. Hence for this embodiment, the solution prepared in step a) of the method of the invention is at least 3 molar in carboxylate/sulphonate, more preferably at least 5 molar and most preferably at least 7 molar. Solubility data available in the literature (for instance the “Merck Index” 13^{th} edition, John Wiley and Sons (2001)) may be used to choose the most suitable cation for a given application. If for instance hyperpolarised acetate is intended to be used as an imaging agent for in vivo MR imaging/chemical shift imaging Cs-acetate or K-acetate are preferably used in the method of the invention since these compounds have a higher solubility in water than for instance NH₄-acetate or Ca-acetate and thus a higher concentrated aqueous solution of acetate can be prepared.

The solution prepared in step a) of the method of the invention further comprises a DNP agent, which is essential in the DNP method. To achieve a high nuclear polarisation level in the carboxylate/sulphonate to be polarised, the DNP agent has to be stable and soluble in the dissolved carboxylate/sulphonate. In this context, stable trityl radicals are the preferred DNP agents and such stable oxygen-based, sulphur-based or carbon-based trityl radicals are for instance described in WO-A-99/35508, WO-A-88/10419, WO-A-90/00904, WO-A-91/12024, WO-A-93/02711, WO-A-96/39367, WO-A-98/39277 and WO-A-2006/011811.

The optimal choice of the DNP agent depends on several aspects. As mentioned before, the DNP agent and the carboxylate/sulphonate must be in intimate contact in order to result in optimal polarisation levels in the carboxylate. Thus, in a preferred embodiment the DNP agent is soluble in the dissolved carboxylate. Suitably, if the carboxylate/sulphonate to be polarised is a lipophilic (hydrophilic) compound, the DNP agent should be lipophilic (hydrophilic) too. If the DNP agent is a trityl radical, lipophilicity or hydrophilicity of said trityl radical can be influenced by choosing suitable lipophilic or hydrophilic residues. Further, the DNP agent has to be stable in presence of the dissolved carboxylate/sulphonate. Hence if the carboxylate/sulphonate contains reactive groups, a DNP agent should be used which is relatively inert towards these reactive groups. From the aforesaid it is apparent that the choice of the DNP agent is highly dependent on the chemical nature and properties of the carboxylate/sulphonate.

In a preferred embodiment, pyruvate is used as a carboxylate in the method of the invention, more preferred ¹³C-pyruvate and most preferred ¹³C₁-pyruvate and the inorganic cation is NH₄⁺, K⁺, Rb⁺ or Cs⁺, preferably K⁺, Rb⁺ or Cs⁺, more preferably Rb⁺ or Cs⁺ and most preferably Cs⁺. In this case, the DNP agent is preferably a trityl radical of the formula (I)

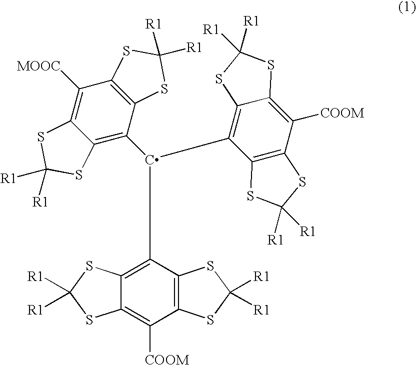

wherein
M represents hydrogen or one equivalent of a cation; and
R1 which is the same or different represents a straight chain or branched C₁-C₆-alkyl group, C₁-C₆-hydroxyalkyl group or a group —(CH₂)ₙ—X—R2, wherein
   n is 1, 2 or 3;
   X is O or S; and
   R2 is a straight chain or branched C₁-C₄-alkyl group.

In a preferred embodiment, M represents hydrogen or one equivalent of a physiologically tolerable cation. The term “physiologically tolerable cation” denotes a cation that is tolerated by the human or non-human animal living body. Preferably, M represents hydrogen or an alkali cation, an ammonium ion or an organic amine ion, for instance meglumine. Most preferably, M represents hydrogen or sodium.

In a further preferred embodiment, R1 is the same, more preferably a straight chain or branched C₁-C₄-alkyl group, most preferably methyl, ethyl or isopropyl or a C₁-C₄-hydroxyalkyl group, most preferably hydroxymethyl or hydroxyethyl.

In a further preferred embodiment, R1 is the same or different, preferably the same and represents —CH₂—OCH₃, —CH₂—OC₂H₅, —CH₂—CH₂—OCH₃, —CH₂—SCH₃, —CH₂—SC₂H₅ or —CH₂—CH₂—SCH₃, most preferably —CH₂—CH₂—OCH₃.

Such trityl radicals may be synthesized as described in detail in WO-A-88/10419, WO-A-90/00904, WO-A-91/12024, WO-A-93/02711, WO-A-96/39367, WO-A-98/39277 and WO-A-2006/011811.

The solution prepared in step a) of the method of the invention may be preferably obtained by dissolving the carboxylate/sulphonate in a suitable solvent or solvent mixture. To this solution, the DNP agent is added and dissolved therein. The DNP agent might be added as a solid or dissolved in a suitable solvent. Preferably, the amount of solvent to dissolve the carboxylate/sulphonates and, if dissolved, the DNP agent, is kept to a minimum. In another preferred embodiment, the DNP agent is dissolved in a suitable solvent and the carboxylate/sulphonate is added to this solution. Intimate mixing of the compounds can be promoted by several means known in the art, such as stirring, vortexing or sonication.

Further, the solution prepared in step a) of the method of the invention optionally comprises a paramagnetic metal ion. The presence of paramagnetic metal ions is preferred since it leads to increased polarisation levels in the carboxylate/sulphonates as explained in detail in PCT/NO06/00449.

The paramagnetic metal ion used in the method of the invention is a paramagnetic metal ion of a lanthanide metal of atomic numbers 58-70 or of a transition metal of atomic numbers 21-29, 42 or 44. Paramagnetic metal ions of one or several different metals may be used, however preferably paramagnetic metal ions of one metal are used. Suitable paramagnetic ions include for instance Cr³⁺, Mn²⁺, Fe³⁺, Fe²⁺, Co²⁺, Ni²⁺, Cu²⁺, Nd³⁺, Gd³⁺, Tb³⁺, Dy³⁺, Er³⁺ and Yb³⁺. In a preferred embodiment the paramagnetic metal ion is chosen from the group consisting of Cr³⁺, Mn²⁺, Fe³⁺, Fe²⁺, Gd³⁺ and Tb³⁺, in a more preferred embodiment from the group consisting of Cr³⁺, Mn²⁺, Fe³⁺ and Gd³⁺.

Suitably, the paramagnetic metal ions are used in chelated form or in the form of their salts. Thus the term “paramagnetic metal ion” denotes salts comprising paramagnetic metal ions as the cation and an anion which is either an organic anion or an inorganic anion. Further, the term “paramagnetic metal ion” also denotes paramagnetic metal ions in chelated form, i.e. so-called paramagnetic chelates. Paramagnetic chelates are complexes of paramagnetic metal ions and a chelating agent.

If the carboxylate/sulphonate to be polarised is intended to be used for solid state NMR, paramagnetic metal ions are preferably used in form of their salts. Suitable salts are for example CrCl₃, MnCl₂, FeCl₂, FeCl₃, GdCl₃ or paramagnetic metal carboxylates/sulphonates, preferably carboxylates/sulphonates which are those that are polarised. Hence if acetate is to be polarised, a paramagnetic metal acetate, for instance Fe(III) acetate can be used as the paramagnetic metal ion. It is of advantage to select a paramagnetic metal salt that is soluble in the solution of the carboxylate/sulphonate and DNP agent. In another embodiment, the paramagnetic metal ions may be added in chelated form.

For liquid state NMR or use as an imaging agent in a living human or animal body, the solid hyperpolarised carboxylate/sulphonate obtained by the method of the invention has to be dissolved or melted to result in a solution or liquid. However, free, i.e. unchelated paramagnetic metal ions in such a solution or liquid dramatically shorten the T₁ relaxation time of the polarised nuclei in the carboxylate/sulphonate, i.e. accelerating the natural decay of the polarisation and thus shortening the time the polarised carboxylate/sulphonate will provide high MR signal intensities. Further, if the carboxylate/sulphonate to be polarised is intended to be used as an imaging agent in a living human or animal body free paramagnetic metal ions often are not or poorly physiologically tolerated and thus have unwanted effects, e.g. toxic effects.

To overcome the aforementioned effects of free paramagnetic metal ions, the paramagnetic metal ions may be used in chelated form, i.e. paramagnetic chelates may be used in the method of the invention. The advantage is that such paramagnetic chelates do not need to be removed from the liquid hyperpolarised carboxylate/sulphonates. However, if the removal of the paramagnetic chelate from the liquid hyperpolarised carboxylate/sulphonate is desired, said removal does not have to be carried out under such high time pressure to avoid T₁ shortening as discussed above. By using a carboxylate/sulphonate according to the method of the invention instead of polarising the free carboxylic acid/sulphonic acid, it is also possible to use a considerably wider range of paramagnetic chelates, most of which would not be stable in a free carboxylic acid/sulphonic acid due to protonation of the nitrogen atoms and carboxylic groups commonly present in the chelating agents.

The aforementioned effects can further be overcome by using paramagnetic metal ions in form of their salts and rapidly removing the paramagnetic metal ions after dissolving or melting the solid hyperpolarised carboxylate/sulphonate. Methods for the removal of paramagnetic metal ions are disclosed later in this application.

In another embodiment, the aforementioned effects can be overcome by using paramagnetic metal ions in form of their salts and adding chelating agents to the dissolution medium the solid hyperpolarised carboxylate/sulphonate is dissolved in to rapidly complex free paramagnetic metal ions. In this case a chelating agent should be chosen that is soluble and stable in the dissolution medium and that rapidly forms a stable complex with the free paramagnetic metal ion.

As stated above, paramagnetic metal ions may be used in the method of the invention in chelated form, i.e. paramagnetic chelates consisting of paramagnetic metal ions and chelating agents.

A variety of chelating agents is known for this purpose. Generally, cyclic and acyclic chelating agents often containing heteroatoms like N, O, P or S may be used with cyclic chelating agents being the preferred ones. Suitable acyclic chelating agents are for instance DTPA and compounds thereof like DTPA-BMA, DTPA-BP, DTPA-BMEA, E013-DTPA, BOPTA and MS-325, EDTA and compounds thereof like EDTA-BMA, DPDP, PLED, HPTA, amides or diamides like TOGDA, cryptands or sulphonates. Suitable cyclic chelating agents are for instance PCTA-[12], PCTP-[12], PCTP-[13], DOTA, DO3A and compounds thereof like HP-DO3A and DO3A-butriol. DOTA, DO3A and compounds thereof are preferred cyclic chelating agents. These chelating agents are known in the art and the skilled artisan is able to find extensive literature describing these chelating agents and their preparation.

In another preferred embodiment, chelating agents are used that are relatively inert chemical entities like for instance fullerenes or zeolites. The use of such chelating agents (encapsulating a paramagnetic metal ion like Gd³⁺) are preferred if the carboxylate/sulphonate to be polarised comprises reactive functional groups that could react with more reactive chelating agents.

In the method of the invention, the paramagnetic chelates may either be monomeric paramagnetic chelates, i.e. chemical entities consisting of a chelating agent and a single paramagnetic metal ion like for instance GdDTPA-BMA or MnDPDP. On the other hand, the paramagnetic chelates may be multimeric paramagnetic chelates, i.e. chemical entities consisting of two or more subunits wherein each subunit consists of a chelating agent and a single paramagnetic metal ion.

As with the DNP agent described before, the carboxylate/sulphonate to be polarised must be in intimate contact with the paramagnetic metal ion as well. In the following, unless otherwise stated or specified, the term “paramagnetic metal ion” is used for both paramagnetic metal ions in form of their salts and paramagnetic chelates. The preparation of the solution in step a) of the method of the invention may be carried out in several ways. In a first embodiment the carboxylate/sulphonate is dissolved in a suitable solvent or suitable solvents to obtain a solution. To this solution, the DNP agent is added and dissolved. The DNP agent might be added as a solid or in solution. Preferably, the amount of solvent(s) to dissolve the DNP agent is kept to a minimum. In a subsequent step, the paramagnetic metal ion is added. The paramagnetic metal ion might be added as a solid or in solution. Again, preferably, the amount of solvent(s) to dissolve the paramagnetic metal ion is kept to a minimum. In another embodiment, the DNP agent and the paramagnetic metal ion are dissolved in a suitable solvent or suitable solvents to form a solution and the carboxylate/sulphonate is added to this solution. In yet another embodiment, the DNP agent (or the paramagnetic metal ion) is dissolved in a suitable solvent or suitable solvents to form a solution and the carboxylate/sulphonate is added to this solution. In a subsequent step the paramagnetic metal ion (or the DNP agent) is added to this solution, either as a solid or dissolved in a suitable solvent or suitable solvents. Preferably, the amount of solvent(s) to dissolve the paramagnetic metal ion (or the DNP agent) is kept to a minimum. Intimate mixing of the compounds can be promoted by several means known in the art, such as stirring, vortexing or sonication.

It is preferred to use a paramagnetic metal ion which is soluble in the solution of the carboxylate/sulphonate and DNP agent. If the carboxylate/sulphonate to be polarised is a lipophilic (hydrophilic) compound and if the paramagnetic metal ion used is a paramagnetic chelate, said chelate should be lipophilic (hydrophilic) too. Lipophilicity or hydrophilicity of paramagnetic chelates can for instance be influenced by choosing chelating agents that comprise suitable lipophilic or hydrophilic residues. It is further preferred that the paramagnetic chelate is stable in presence of the carboxylate/sulphonate since dissociation (dechelation) of the paramagnetic chelate will lead to free paramagnetic ions with detrimental consequences on the polarisation decay and hence polarisation level in a liquefied hyperpolarised carboxylate/sulphonate as described above, unless the free paramagnetic metal ions are rapidly removed after the solid hyperpolarised carboxylate/sulphonate has been liquefied. If the carboxylate/sulphonate to be polarised contains reactive groups a paramagnetic metal ion should be used which is relatively inert towards these reactive groups. From the aforesaid it is apparent that the choice of the paramagnetic metal ion is highly dependent on the chemical nature of the carboxylate/sulphonate and its final use (solid NMR, liquid NMR or MR imaging agent/chemical shift agent for in vivo use).

If a trityl radical is used as DNP agent, a suitable concentration of such a trityl radical is 5 to 25 mM, preferably 10 to 20 mM in the solution prepared in step a). If a paramagnetic metal ion is present in the solution prepared in step a), a suitable concentration of such a paramagnetic metal ion is 0.1 to 6 mM (metal ion) and a concentration of 0.5 to 4 mM is preferred.

In the method of the invention, after having prepared the solution in step a), said solution is frozen in step b). This can be done by methods known in the art, e.g. by freezing the solution in a freezer, in liquid nitrogen—preferably as “beads” obtained by adding drops of the solution prepared in step a) into liquid nitrogen—or by simply placing it in a suitable container and inserting it into the DNP polariser, where liquid helium will freeze it. Solutions containing a high concentration of carboxylate/sulphonate have a low freezing point and freezing such solutions in a freezer at a temperature of about −18° C. will be sufficient to obtain a frozen solution.

If a paramagnetic metal ion is present in the solution said solution may be degassed before freezing. Degassing may be achieved by bubbling helium gas through the solution (e.g. for a time period of 2-15 min) but can be effected by other known common methods.

In step c) of the method of the invention, dynamic nuclear polarisation (DNP) is carried out on the frozen solution, said dynamic nuclear polarisation resulting in a frozen solution comprising the hyperpolarised carboxylate or the hyperpolarised sulphonate or mixtures thereof.

The DNP technique is for instance described in WO-A-98/58272 and in WO-A-01/96895, both of which are included by reference herein. Generally, a moderate or high magnetic field and a very low temperature are used in the DNP process, e.g. by carrying out the DNP process in liquid helium and a magnetic field of about 1 T or above. Alternatively, a moderate magnetic field and any temperature at which sufficient polarisation enhancement is achieved may be employed. In a preferred embodiment, the DNP process is carried out in liquid helium and a magnetic field of about 1 T or above. Suitable polarisation units (=polarisers) are for instance described in WO-A-02/37132. In a preferred embodiment, the polariser comprises a cryostat and polarising means, e.g. a microwave chamber connected by a wave guide to a microwave source in a central bore surrounded by magnetic field producing means such as a superconducting magnet. The bore extends vertically down to at least the level of a region P near the superconducting magnet where the magnetic field strength is sufficiently high, e.g. between 1 and 25 T, for polarisation of NMR active nuclei to take place. The bore for the probe (=the frozen solution to be polarised) is preferably sealable and can be evacuated to low pressures, e.g. pressures in the order of 1 mbar or less. A probe introducing means such as a removable transporting tube can be contained inside the bore and this tube can be inserted from the top of the bore down to a position inside the microwave chamber in region P. Region P is cooled by liquid helium to a temperature low enough to for polarisation to take place, preferably temperatures of the order of 0.1 to 100 K, more preferably 0.5 to 10 K, most preferably 1 to 5 K. The probe introducing means is preferably sealable at its upper end in any suitable way to retain the partial vacuum in the bore. A probe-retaining container, such as a probe-retaining cup, can be removably fitted inside the lower end of the probe introducing means. The probe-retaining container is preferably made of a light-weight material with a low specific heat capacity and good cryogenic properties such, e.g. KelF (polychlorotrifluoro-ethylene) or PEEK (polyetheretherketone) and it may be designed in such a way that it can hold more than one probe.

The probe (liquid or already frozen) is inserted into the probe-retaining container, submerged in the liquid helium and irradiated with microwaves, preferably at a frequency of about 94 GHz at 200 mW. The level of polarisation may be monitored by for instance acquiring solid state NMR signals of the probe during microwave irradiation. Generally, a saturation curve is obtained in a graph showing NMR signal vs. time. Hence it is possible to determine when the optimal polarisation level is reached. A solid state NMR measurement, for instance a solid state ¹³C-NMR measurement suitably consists of a simple pulse-acquire NMR sequence using a low flip angle. The signal intensity of the hyperpolarised carboxylate/sulphonates in the NMR spectrum is compared with signal intensity of the carboxylate/sulphonates in an NMR spectrum acquired before the dynamic nuclear polarisation process. The level of polarisation is then calculated from the ratio of the signal intensities of before and after DNP.

If the hyperpolarised carboxylate/sulphonate is intended for use as MR imaging agent/chemical shift agent or in liquid state NMR spectroscopy, the frozen solution containing the hyperpolarised carboxylate/sulphonate needs to be transferred from a solid state to a liquid state, i.e. liquefied. Hence the method of the invention may contain a further step d) wherein the frozen solution obtained in step c) is liquefied. This can be done by dissolving the frozen solution obtained in step c) in an appropriate solvent or solvent mixture. An aqueous carrier, preferably a physiologically tolerable and pharmaceutically accepted aqueous carrier like water, a buffer solution or saline is suitably used as a solvent, preferably if the hyperpolarised carboxylate/sulphonate is intended for use as MR imaging agent/chemical shift agent in vivo. Further, non aqueous solvents or solvent mixtures may be used, for instance solvents like DMSO or methanol or mixtures comprising an aqueous carrier and a non aqueous solvent, for instance mixtures of DMSO and water or methanol and water. Alternatively, the frozen solution can be liquefied in step d) by melting it.

Dissolution is preferred and the dissolution process of a frozen solution containing a DNP polarised compound and suitable devices therefore are described in detail in WO-A-02/37132. The melting process and suitable devices for the melting are for instance described in WO-A-02/36005.

In a preferred embodiment, the frozen solution obtained in step c) and comprising the hyperpolarised carboxylate/sulphonate is dissolved in water.

By liquefying the frozen solution after the dynamic nuclear polarisation, a liquid comprising hyperpolarised carboxylate or hyperpolarised sulphonate or mixtures thereof is obtained, wherein the hyperpolarised carboxylate or hyperpolarised sulphonate comprises an inorganic cation from the group consisting of NH₄⁺, K⁺, Rb⁺, Cs⁺, Ca²⁺, Sr²⁺ and Ba²⁺.

In a subsequent step, the DNP agent and the optionally present paramagnetic metal ion may be removed from the liquid. If the hyperpolarised carboxylate/sulphonate is intended to be used as MR imaging agent/chemical shift agent in vivo, the DNP agent, which is preferably a trityl radical and the paramagnetic metal ion are preferably removed from the liquid.

Methods useful to partially, substantially or completely remove the trityl radical and the paramagnetic metal ion are known in the art. Generally, the methods applicable depend on the nature of the trityl radical and the paramagnetic metal ion. Upon dissolution of the frozen solution and depending on the chemical nature of the solvent(s) used, the trityl radical and/or the paramagnetic metal ion may precipitate and thus may easily be separated from the liquid comprising the hyperpolarised carboxylate/sulphonates by filtration.

If no precipitation occurs, the trityl radical and the paramagnetic metal ion may be removed by chromatographic separation techniques, e.g. liquid phase chromatography like reversed phase chromatography, ion exchange chromatography, (solid phase) extraction or other chromatographic separation methods known in the art. In general, it is preferred to use a method where both the trityl radical and the paramagnetic metal ion can be removed in one step as polarisation of the carboxylate/sulphonate in the liquid decays due to T₁ relaxation. The faster and the more efficient unwanted compounds are removed from the liquid the higher the polarisation level retained in the carboxylate/sulphonate. Hence it is of benefit to select a trityl radical and a paramagnetic metal ion which have similar chemical properties, e.g. which both are lipophilic or hydrophilic chemical compounds or have certain functional groups in common. If for instance a lipophilic trityl radical and a lipophilic paramagnetic chelate are used, both compounds could be removed by reversed phase liquid chromatography.

If free paramagnetic metal ions are present in the liquid (e.g. due to the use of a paramagnetic metal salt), these ions are preferably removed by using a cation exchange column or ionic imprinted resins as described by O. Vigneau et al., Anal. Chim. Acta 435(1), 2001, 75-82. Another possible method is nano-filtration by selective complexation of free paramagnetic metal ions onto a charged organic membrane, as disclosed by A. Sorin et al., J. Membrane Science 267(1-2), 2005, 41-49. Further, free paramagnetic metal ions may be removed by affinity chromatography in analogy to what is disclosed by S. Donald et al. J. Inorg. Biochem. 56(3), 1994, 167-171.

As trityl radicals have a characteristic UV/visible absorption spectrum, it is possible to use UV/visible absorption measurement as a method to check for their presence in the liquid after their removal. In order to obtain quantitative results, i.e. the concentration of the trityl radical present in the liquid, the optical spectrometer can be calibrated such that absorption at a specific wavelength from a sample of the liquid yields the corresponding trityl radical concentration in the sample. Removal of the trityl radical is especially preferred if the liquid comprising the hyperpolarised carboxylate/sulphonate is used as imaging agent/chemical shift agent in vivo.

Fluorescence or UV/visible absorption measurement can be used as a method to check for the presence of paramagnetic chelates, provided that the chelates contain a (strong) chromophore. Another way to check for the presence of paramagnetic chelates is electrochemical detection, provided an electroactive moiety is present in the chelate.

If paramagnetic metal salts were used in the preparation of the solution in step a) of the method of the invention, fluorescence measurements may be used to check for free paramagnetic metal ions after their removal from the liquid. If for instance a Gd³⁺-salt is used, fluorescence with an excitation wavelength of 275 nm and monitoring of emission at 314 nm may be used as a method to detect free Gd³⁺ with high specificity. Further, free Gd³⁺ can be detected by visible absorbance at 530-550 nm following complexation with the colorimetric agent PAR (4-(2-pyridylazo) resorcinol). Further colorimetric agents suitable for other paramagnetic metal ions are known in the art and can be used in the same way.

In the following a preferred embodiment of the method according to the invention is described. In this preferred embodiment, the solution prepared in step a) of the method of the invention is an aqueous solution that comprises ¹³C-pyruvate, preferably ¹³C₁-pyruvate or ¹³C-bicarbonate, and a cation from the group consisting of K⁺, Rb⁺ and Cs⁺, preferably Cs⁺. For simplification reasons, the preferred embodiment is illustrated for Cs-¹³C-pyruvate hereinafter. The aqueous solution further comprises a trityl radical, preferably a trityl radical of formula (I) and either a paramagnetic chelate comprising Gd³⁺ (Gd-chelate) or a Gd³⁺-salt (Gd-salt) like GdCl₃ as a paramagnetic metal ion. Cs-¹³C-pyruvate is prepared by cation exchange of a commercially available ¹³C-pyruvate salt, preferably Na-¹³C-pyruvate, by passing an aqueous solution of Na-¹³C-pyruvate through a ion exchange column or cartridge which was charged using an aqueous solution of a soluble Cs-salt like CsCl. The so prepared Cs-¹³C-pyruvate is freeze dried. The aqueous solution used in the method of the invention is prepared by dissolving each the trityl radical and the Gd-chelate or Gd-salt in a minimum of water. An aqueous solution of Cs-¹³C-pyruvate is prepared said aqueous solution is preferably at least 5 molar in pyruvate. This aqueous solution is then combined with the dissolved trityl radical and the dissolved Gd-chelate or Gd-salt. The resulting aqueous solution is frozen in step b), for instance in a freezer and then used for dynamic nuclear polarisation in step c). In step d), after the DNP process, the frozen solution comprising the hyperpolarised Cs-¹³C-pyruvate is dissolved in an aqueous carrier, preferably in water and thus a liquid comprising hyperpolarised Cs-¹³C-pyruvate is obtained. If a Gd³⁺-salt has been used as paramagnetic metal ion, it is important to remove Gd³⁺ ions from the dissolved hyperpolarised Cs-¹³C-pyruvate as quickly as possible, especially if the hyperpolarised Cs-¹³C-pyruvate is going to be used as MR imaging agent/chemical shift agent in vivo. Suitable methods are the removal by using a cation exchange column or ionic imprinted resins as disclosed by O. Vigneau et al., Anal. Chim. Acta 435(1), 2001, 75-82. Another possible method is nano-filtration by selective complexation of free Gd³⁺ onto a charged organic membrane, as disclosed by A. Sorin et al., J. Membrane Science 267(1-2), 2005, 41-49. Further, free Gd³⁺ may be removed by affinity chromatography as disclosed by S. Donald et al. J. Inorg. Biochem. 56(3), 1994, 167-171.

If a Gd-chelate has been used as paramagnetic metal ion, and a trityl radical of formula (I), the chelate may be removed by using reversed phase liquid chromatography, which allows the simultaneous removal of the trityl radical of formula (I).

Suitable methods to check for residual free Gd³⁺, Gd-chelate and trityl radical of formula (I) in the liquid comprising the hyperpolarised Cs-¹³C-pyruvate are described on page 25/26.

If the liquid comprising the hyperpolarised Cs-¹³C-pyruvate is intended to be used as MR imaging agent/chemical shift agent in vivo, it may be desirable to exchange Cs⁺ by other types of cations which are known to be physiologically tolerable like sodium or meglumine and thus using for instance hyperpolarised sodium-¹³C-pyruvate as MR imaging agent/chemical shift agent in vivo. Methods to do such a cation exchange are known in the art and it is preferred to use a fast method since polarisation of the hyperpolarised ¹³C-pyruvate decays over time. In a preferred embodiment the cation exchange is carried out using a cation exchange column or cartridge which is charged with the desired cation, e.g. sodium or meglumine and the liquid comprising the hyperpolarised Cs-¹³C-pyruvate is passed through this column or cartridge. Suitably the total time of the cation exchange is less than 10 s, preferably less than 7 s and more preferably less than 5 s.

A liquid comprising hyperpolarised ¹³C-pyurvate produced according to the method of the invention may be used as a “conventional” MR imaging agent, i.e. providing excellent contrast enhancement for anatomical imaging. A further advantage of liquid hyperpolarised ¹³C-pyurvate produced according to the method of the invention is that pyruvate is an endogenous compound which is well tolerated by the human or non-human animal body, even in higher concentrations. As a precursor in the citric acid cycle, pyruvate plays an important metabolic role in the human/mammalian body where it is converted into different compounds: its transamination results in alanine; via oxidative decarboxylation pyruvate is converted into acetyl-CoA and bicarbonate, the reduction of pyruvate results in lactate and its carboxylation in oxaloacetate.

Additionally, the metabolic conversion of hyperpolarised ¹³C-pyruvate to hyperpolarised ¹³C-lactate, hyperpolarised ¹³C-bicarbonate (in the case of ¹³C₁-pyruvate, ¹³C_{1,2}-pyruvate or ¹³C_{1,2,3}-pyruvate only) and hyperpolarised ¹³C-alanine can be used for in vivo MR studying of metabolic processes in the human body. ¹³C-pyruvate has a T₁ relaxation in human full blood at 37° C. of about 42 s, however, the conversion of hyperpolarised ¹³C-pyruvate to hyperpolarised ¹³C-lactate, hyperpolarised ¹³C-bicarbonate and hyperpolarised ¹³C-alanine has been found to be fast enough to allow signal detection from the ¹³C-pyruvate parent compound and its metabolites. The amount of alanine, bicarbonate and lactate is dependent on the metabolic status of the tissue under investigation. The MR signal intensity of hyperpolarised ¹³C-lactate, hyperpolarised ¹³C-bicarbonate and hyperpolarised ¹³C-alanine is related to the amount of these compounds and the degree of polarisation left at the time of detection, hence by monitoring the conversion of hyperpolarised ¹³C-pyruvate to hyperpolarised ¹³C-lactate, hyperpolarised ¹³C-bicarbonate and hyperpolarised ¹³C-alanine it is possible to study metabolic processes in vivo in the human or non-human animal body by using non-invasive MR imaging.

It has been found that the MR signal amplitudes arising from the different pyruvate metabolites vary depending on the tissue type. The unique metabolic peak pattern formed by alanine, lactate, bicarbonate and pyruvate can be used as fingerprint for the metabolic state of the tissue under examination and thus allows for the discrimination between healthy tissue and tumour tissue. This makes the composition according to the invention an excellent agent for in vivo MR tumour imaging. The use of pyruvate for tumour imaging has been described in detail in WO-A-2006/011810.

Further, the use of hyperpolarised ¹³C-pyruvate for cardiac imaging has been described in WO-A-2006/054903.

Another aspect of the invention is a composition comprising a carboxylate or a sulphonate or mixtures thereof wherein said carboxylate or sulphonate comprises an inorganic cation from the group consisting of NH₄⁺, K⁺, Rb⁺, Cs⁺, Ca²⁺, Sr²⁺ and Ba²⁺, a DNP agent, preferably a trityl radical and optionally a paramagnetic metal ion.

Preferably, the composition comprises a carboxylate, i.e. a certain carboxylate or several different carboxylates, preferably an endogenous carboxylate and more preferably an endogenous carboxylate that plays a role in a metabolic process in the human or non-human animal body. In a further preferred embodiment, said carboxylate is a ¹³C enriched carboxylate, preferably enriched at a carboxyl atom, a carbonyl atom or a quaternary C-atom.

Preferred carboxylates are malate, acetate, fumarate, lactate, citrate, pyruvate, bicarbonate, malonate, carbonate, succinate, oxaloacetate, α-ketoglutarate, 2-oxobutanoate, 2-oxo-5-methylpentanoate, γ-carboxyglutamate, pyridine-2,3-dicarboxylate and isocitrate. Most preferred carboxylates are bicarbonate, fumarate, carbonate, acetate, lactate, 2-oxobutanoate, 2-oxo-5-methylpentanoate, γ-carboxyglutamate, pyridine-2,3-dicarboxylate and pyruvate.

Preferred inorganic cation are NH₄⁺, K⁺, Rb⁺ or Cs⁺, more preferred K⁺, Rb⁺ or Cs⁺ and most preferred Rb⁺ or Cs⁺.

In a preferred embodiment the composition of the invention is dissolved in a solvent or solvent mixture to result in a solution, preferably an aqueous solution. Alternatively, the solution is a non aqueous solution. Suitable solvent or solvent mixtures for such non aqueous solutions are or comprise for instance DMSO or methanol. In yet another embodiment the solution comprises a mixture of a solvent, like for instance DMSO and/or methanol and water. A preferred solvent is water.

In yet another preferred embodiment the solution is at least 3 molar in carboxylate or sulphonate or mixtures thereof, more preferably at least 5 molar and most preferably at least 7 molar.

In yet another preferred embodiment of the composition according to the invention the DNP agent is a stable oxygen-based, sulphur-based or carbon-based trityl radical and/or the composition comprises a paramagnetic metal ion, preferably a paramagnetic metal ion of a lanthanide metal of atomic numbers 58-70 or of a transition metal of atomic numbers 21-29, 42 or 44. Suitably, the paramagnetic metal ion is in chelated form or in form of a salt.

The composition of the invention can be used in dynamic nuclear polarisation.

Definitions and preferred embodiments described for the method of the invention on pages 7 to 20 of this application apply likewise to the composition described above.

Yet another aspect of the invention is a composition comprising a hyperpolarised carboxylate or hyperpolarised sulphonate or mixtures thereof wherein said hyperpolarised carboxylate or hyperpolarised sulphonate comprises an inorganic cation from the group consisting of NH₄⁺, K⁺, Rb⁺, Cs⁺, Ca²⁺, Sr²⁺ and Ba²⁺.

In a preferred embodiment, said composition comprises a hyperpolarised carboxylate, i.e. a certain carboxylate or several different carboxylates, preferably a hyperpolarised endogenous carboxylate and more preferably a hyperpolarised endogenous carboxylate that plays a role in a metabolic process in the human or non-human animal body. Preferably the hyperpolarised carboxylate is a ¹³C enriched carboxylate, preferably enriched at a carboxyl atom, a carbonyl atom or a quaternary C-atom.

In another preferred embodiment the hyperpolarised carboxylate is a hyperpolarised carboxylate from the group consisting of malate, acetate, fumarate, lactate, citrate, pyruvate, bicarbonate, malonate, carbonate, succinate, oxaloacetate, α-ketoglutarate, 2-oxobutanoate, 2-oxo-5-methylpentanoate, γ-carboxyglutamate, pyridine-2,3-dicarboxylate and isocitrate. Most preferred hyperpolarised carboxylates are hyperpolarised carboxylates from the group consisting of bicarbonate, fumarate, carbonate, acetate, lactate, 2-oxobutanoate, 2-oxo-5-methylpentanoate, γ-carboxyglutamate, pyridine-2,3-dicarboxylate and pyruvate.

Preferably the inorganic cation is NH₄⁺, K⁺, Rb⁺ or Cs⁺, preferably K⁺, Rb⁺ or Cs⁺ and more preferably Rb⁺ or Cs⁺.

In another embodiment the composition further comprises a DNP agent and optionally a paramagnetic metal ion and is obtained by dynamic nuclear polarisation.

In yet another embodiment the composition comprising a hyperpolarised carboxylate or sulphonate or mixtures thereof wherein said hyperpolarised carboxylate or sulphonate comprises an inorganic cation from the group consisting of NH₄⁺, K⁺, Rb⁺, Cs⁺, Ca²⁺, Sr²⁺ and Ba²⁺ is dissolved in a solvent or solvents.

Preferably the solvent is an aqueous carrier, more preferably a physiologically tolerable and pharmaceutically accepted aqueous carrier like water, a buffer solution or saline or a non aqueous solvent. In another preferred embodiment, the solvent is a non aqueous solvent. If more than one solvent is used, said solvents may for instance be mixtures of DMSO or methanol or solvent mixtures comprising an aqueous carrier and a non aqueous solvent, for instance mixtures of DMSO and water or methanol and water.

In a preferred embodiment, the composition comprising a hyperpolarised carboxylate or sulphonate or mixtures thereof wherein said hyperpolarised carboxylate or sulphonate comprises an inorganic cation from the group consisting of NH₄⁺, K⁺, Rb⁺, Cs⁺, Ca²⁺, Sr²⁺ and Ba²⁺ is dissolved in an aqueous carrier. In a further preferred embodiment, said dissolved composition is used as an MR imaging agent/chemical shift agent in vivo. The inorganic cation may optionally be exchanged by a cation which is very well tolerated in the living human or non-human body, for instance meglumine or sodium cations.

Again definitions and preferred embodiments described for the method of the invention on pages 7 to 21 of this application apply likewise to the composition described above.

### EXAMPLES

### Example 1

### Preparation of a Caesium Charged Ion Exchange Column

A Varian Bond Elution SCX chromatography column (60 ml, 10 g, 8.7 meq) was rinsed with one column volume methanol followed by one column volume of water. One column volume of an aqueous caesium chloride solution (0.8 M, 48 mmol) was allowed to slowly run through the column. The first part of the eluate was strongly acidic (pH 0) but pH rose during the ion exchange process. The last few ml of the eluate had a pH of 4-5. After completed ion exchange the column was rinsed with 2 column volumes of water.

### Example 2

### Preparation of Caesium Pyruvate

Sodium ¹³C₁-pyruvate (425 mg, 3.8 mmol) was dissolved in 20 ml water. The resulting solution was allowed to run through the wet column of Example 1. The eluate (first eluate) was collected. The column was rinsed with 20 ml of water and the eluate from the rinsing process was collected and combined with the first eluate. The combined eluates were freeze-dried. A total of 0.9 g caesium pyruvate as an off-white salt was obtained.

### Analysis:

¹³³Cs-NMR analysis: the integral of a one pulse experiment was compared to a standard caesium solution and the purity of the obtained caesium pyruvate was calculated to be 97-98%

²³Na-NMR analysis: only a very weak sodium signal could be detected

¹H-NMR analysis revealed a water content of less than 1 mole/mole pyruvate. The methyl group of pyruvate resonated at 2.2 ppm. In addition to this, resonance peaks at 1.3 ppm, present also in a solution of the purchased sodium pyruvate used in Example 2, and minor peaks at 1.4 ppm and 3.0-3.2 ppm, appeared. These peaks originate from the hydrate of pyruvate (1.3 ppm) and from parapyruvate (1.2 and 3.0-3.2 ppm). The amount of parapyruvate in the preparation was less than 4%.

### Example 3

### Dynamic Nuclear Polarisation of an Aqueous Solution Containing Caesium Pyruvate and a Trityl Radical as the DNP Agent

Caesium ¹³C₁-pyruvate (99 mg, 0.45 mmol) was dissolved in 16 μl water. 16 μl of a solution of the trityl radical tris-(8-carboxy-2,2,6,6-tetra (1-hydroxyethyl) benzo-[1,2-d:4,5d′]bis(1,3) dithiole 4-yl)methyl sodium salt which was prepared as described in example 7 of WO-A-98/39277 in water (67.3 mM) was added to result in an aqueous solution being 14.5 mM in trityl radical. From this solution 70 μl which contained 94 mg/95% of the caesium pyruvate thus being 7 molar in pyruvate were transferred to a probe cup and inserted in a DNP polariser. The frozen probe was polarised under DNP conditions at 1.2 K in a 3.35 T magnetic field under microwave irradiation (93.950 GHz). After 2 hours, the polarisation was stopped.

After the polarisation had been stopped, the frozen probe was dissolved in 7 ml water containing 100 mg/l EDTA and liquid state polarisation of the ¹³C nuclei in the hyperpolarised caesium pyruvate was determined by liquid state ¹³C-NMR at 400 MHz to be 15%.

### Example 4

### Synthesis of the Gd-chelate of 1,3,5-Tris-(N-(DO3A-acetamido)-N-methyl-4-amino-2-methyl-phenyl)-[1,3,5]triazinane-2,4,6-trione (10)

### 4a) Preparation of 2-Methyl-4-nitrophenylisocyanate (1)

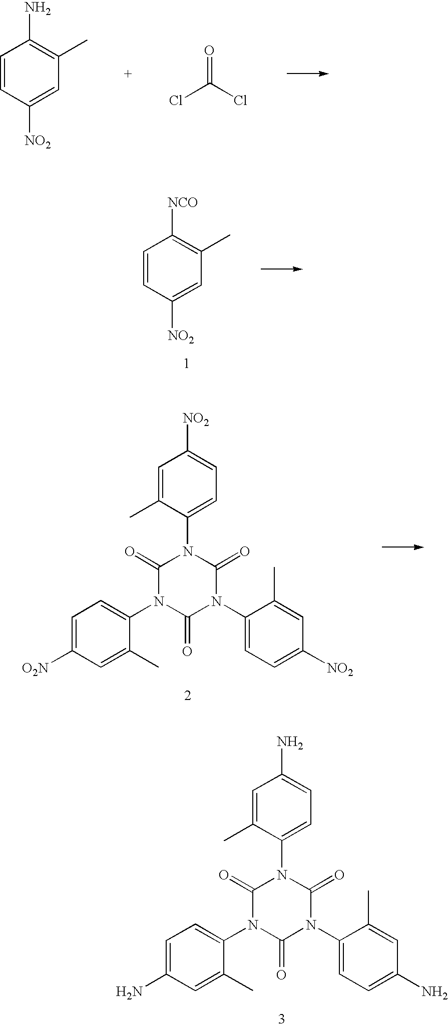

2-Methyl-4-nitroaniline (35.0 g, 230 mmol) was dissolved in ethyl acetate (400 ml) and cooled to 0° C. Phosgene (180 ml, 20% in toluene) was added drop wise over 30 min, precipitation of a white salt followed instantly. After the last addition the temperature was allowed to slowly rise to room temperature, and then the reaction mixture was brought to reflux (˜100° C.). It was refluxed for 2 h 30 min, after which 200 ml of solvent was distilled off before the temperature was lowered to 80° C. and phosgene (140 ml, 20% in toluene) was added drop wise. After the last addition the reaction solution was refluxed for 3 hours, allowed to cool to room temperature and concentrated to dryness. The brown/yellow material was dissolved in diethyl ether (250 ml), filtered and concentrated to give a pale brown powder (36 g, 88%).

### 4b) Preparation of 1,3,5-Tris-(4-nitro-2-methyl-phenyl)-[1,3,5]triazinane-2,4,6-trione (2)

To 2-methyl-4-nitrophenylisocyanate (36.0 g) in a 250 ml flask was added DMSO (50 ml) and the flask was sealed with a glass stopper which was kept in place with a plastic clip. The flask was immediately lowered into an oil bath heated to 85° C. and the dark brown reaction solution was heated for 16 h 30 min. The oil bath was removed and the reaction solution was allowed to cool to room temperature before being poured into water (800 ml), sonicated, and the precipitate was filtered off. The filter cake was added to ethanol (500 ml) and was refluxed for 4 hours, then allowed to cool to room temperature and the product was filtered off to give an off-white powder (28.1 g, 78%).

### 4c) Preparation of 1,3,5-Tris-(4-amino-2-methyl-phenyl)-[1,3,5]triazinane-2,4,6-trione (3)

1,3,5-tris-(4-nitro-2-methyl-phenyl)-[1,3,5]triazinane-2,4,6-trione (2.86 g, 5.4 mmol) was dissolved in THF (70 ml). HCl (4.5 ml, 6M), water (18 ml) and Pd/C (0.6 g, 10%) was added. The reaction vessel was evacuated and filled with argon in three cycles before hydrogenated on a Parr hydrogenation apparatus (60 psi). After 2 hours the excess hydrogen was evacuated with a membrane pump and the Pd/C (10%) was filtered off. The clear reaction solution was concentrated until no more THF remained and the pH adjusted to 7 with NaHCO₃ (−3.7 g). The aqueous phase was extracted with ethyl acetate (3×100 ml) and the combined organic phases were dried with MgSO₄, filtered and concentrated to give a brown powder. The crude product was recrystallized from methanol to give the product as an off-white powder (1.9 g, 80%).

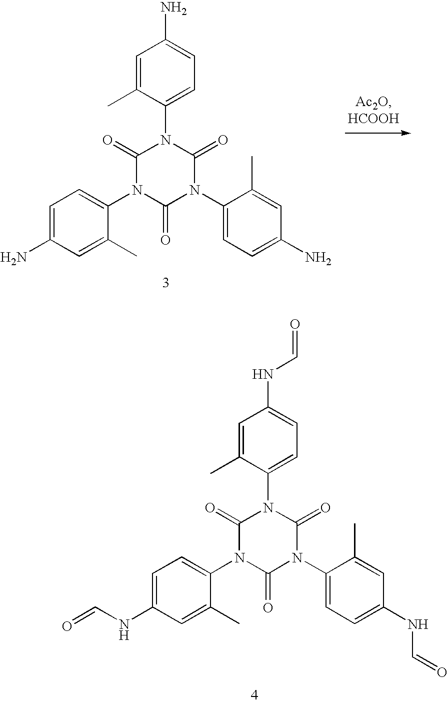

### 4d) Preparation of 1,3,5-Tris-(4-formamido-2-methyl-phenyl)-[1,3,5]triazinane-2,4,6-trione (4)

Formic acid (175 ml) was put in an ice-cooled 500 ml round-bottom flask. Acetic anhydride (15 ml, 0.16 mol) was added and the yellow solution was stirred under argon for 1 h at 0° C. The triamine 3 (8.7 g, 0.020 mol) was added to this solution and the ice bath was removed. After stirring under argon at room temperature for 30 minutes HPLC showed complete reaction. The solvent was removed in vacuo and the brown, sticky residue was suspended in H₂O and filtered off. It was then washed thoroughly with H₂O to make sure all acid was removed. The product was a pale-brown solid (10.2 g, 99%).

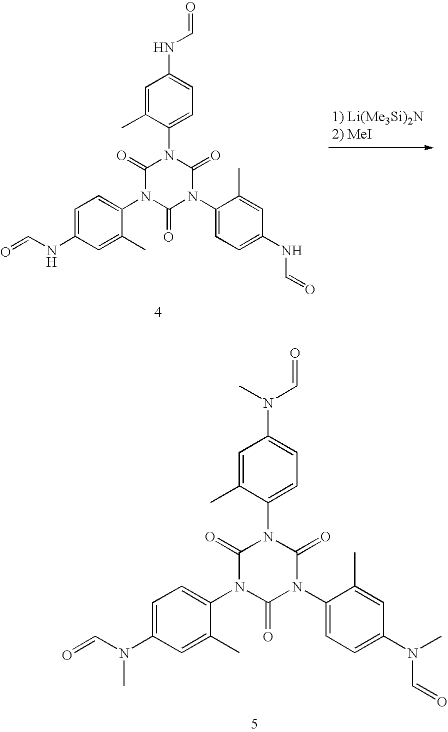

### 4e) Preparation of 1,3,5-Tris-(N-formyl-N-methyl-4-amino-2-methyl-phenyl)-[1,3,5]triazinane-2,4,6-trione (5)

All glassware was carefully dried in oven and DMF was dried over 4 Å molecular sieves. Li(Me₃Si)₂N (116 ml, 0.116 mol, 1 M in hexane) was added to a DMF-solution (115 ml) of 4 (10.2 g, 0.0193 mol) in 500 ml round-bottom flask. The reaction mixture, which turned from a light brown solution to a brick-red slurry, was stirred under argon for 1 h. Methyl iodide (12.2 ml, 0.196 mol) was added and the reaction mixture was stirred for 2 h or until complete methylation could be shown on HPLC. The hexane was then removed on rotary evaporator and the residue was poured into a solution of NaH₂PO₄ (1300 ml, 100 mM) under vigorous stirring. The precipitate of 5 formed was filtered off as a pale solid (6.7 g, 60%).

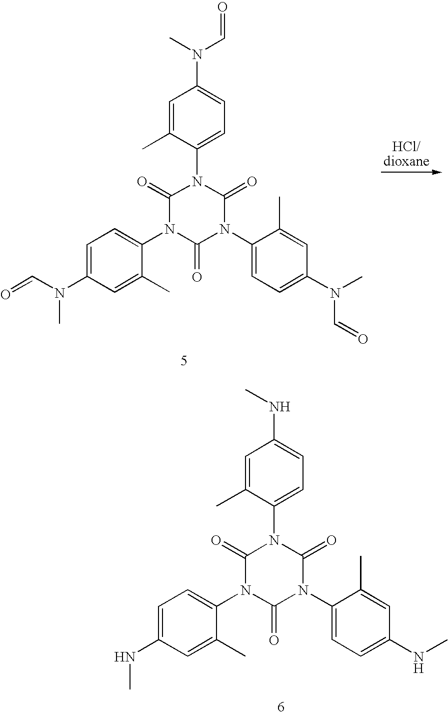

### 4f) Preparation of 1,3,5-Tris-(N-methyl-4-amino-2-methyl-phenyl)-[1,3,5]triazinane-2,4,6-trione (6)

Dioxane (52 ml), HCl (52 ml, 6 M) and 5 (6.5 g, 11 mmol) were mixed in a 250 ml round-bottom flask to form a pale slurry. The reaction mixture was heated to reflux for 30 minutes under argon. The now yellow solution was allowed to cool to room temperature and solvents were then removed on a rotary evaporator. The orange residue was then dissolved in 500 ml H₂O and neutralized with a solution of NaHCO₃ (sat.) under vigorous stirring. The precipitate formed was filtered off and washed several times with H₂O giving a pale solid (4.7 g, 84%).

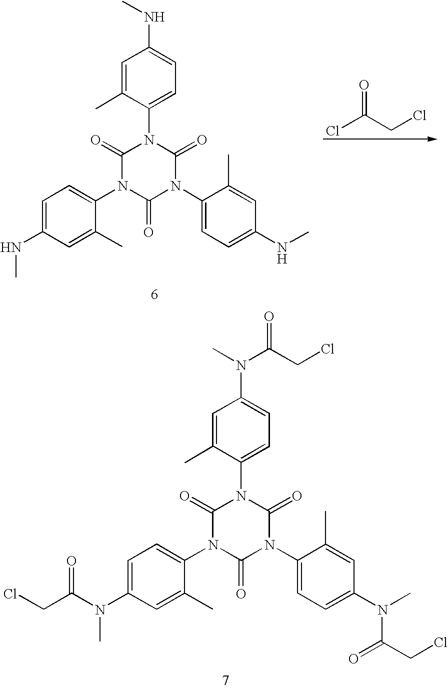

### 4g) Preparation of 1,3,5-Tris-(N-chloroacetyl-N-methyl-4-amino-2-methyl-phenyl)-[1,3,5]triazinane-2,4,6-trione (7)

In a 100 ml round-bottom flask 6 (4.6 g, 9.5 mmol) was dissolved in DMA (15 ml) and chloroacetyl chloride (2.6 ml, 33 mmol) was added under stirring at 0° C. The reaction was stirred under argon at RT for 30 min or until HPLC showed complete chloroacetylation. The slurry was then poured into a large beaker with water (500 ml) under vigorous mechanical stirring. The precipitate formed was filtered off and dried in vacuo at 0.3 mbar (6.3 g). The pale solid was dissolved in 70 ml acetonitrile and poured into 500 ml H₂O under vigorous mechanical stirring. The precipitate formed was filtered off and left to dry in a desiccator (6.1 g, 89%).

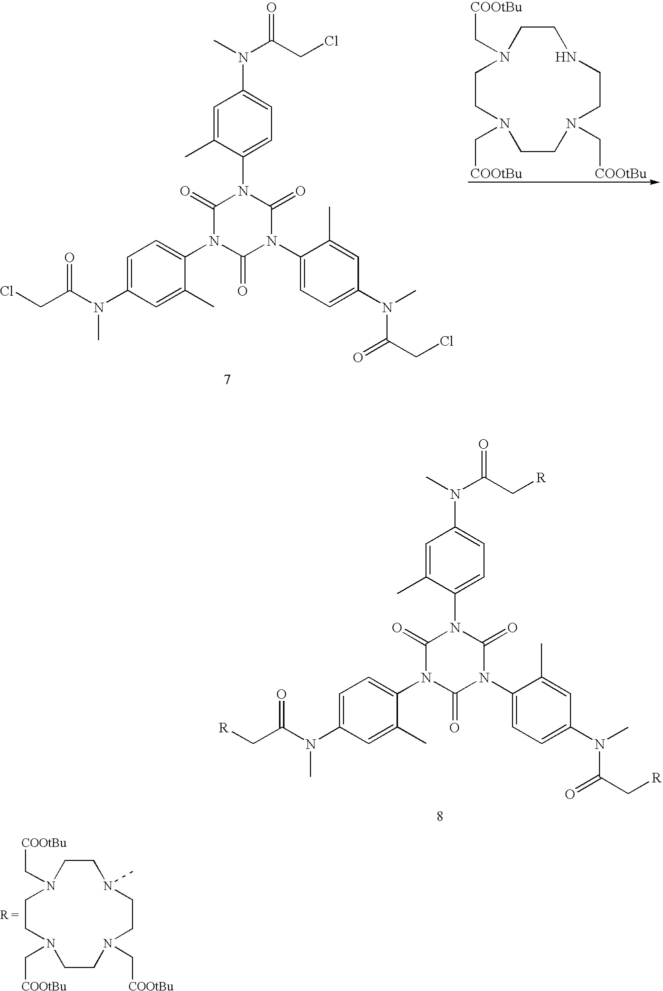

### 4h) Preparation of 1,3,5-Tris-(N-(DO3A t-butylester-acetamido)-N-methyl-4-amino-2-methyl-phenyl)-[1,3,5]triazinane-2,4,6-trione (8)

In a 50 ml round-bottom flask, 7 (0.50 g, 0.70 mmol) was suspended together with DO3A t-butyl ester (2.5 g, 4.2 mmol), diisopropylethylamine (910 μl, 5.2 mmol) and acetonitrile (15 ml). After sonication the reaction mixture was stirred at 75° C. under argon until LC/MS showed complete coupling. The solvents were then removed on rotary evaporator and the crude product (2.9 g) was used in the subsequent reaction.

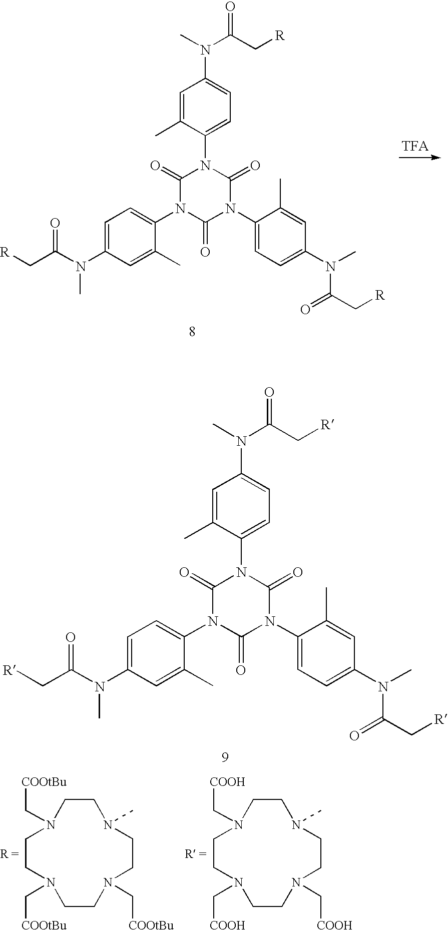

### 4i) Preparation of 1,3,5-Tris-(N-(DO3A-acetamido)-N-methyl-4-amino-2-methyl-phenyl)-[1,3,5]triazinane-2,4,6-trione (9)

The crude product of 8 (1.9 g) was dissolved in TFA (130 ml) and CH₂Cl₂ (130 ml) and was stirred at 50° C. under argon. The solution was stirred for 1 h or until LC/MS showed complete deprotection. The solvents were then removed on rotary evaporator and the residue was dried in vacuo overnight. The crude product (2.4 g) was then used in the final step.

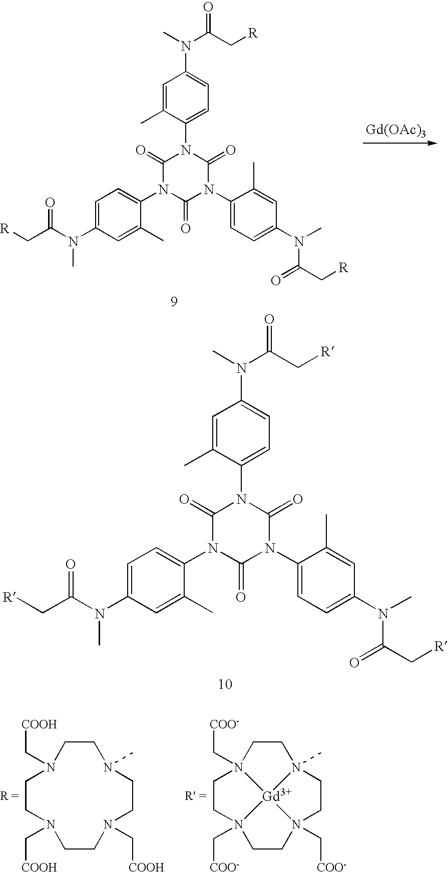

### 4j) Preparation of gadolinium chelate of 1,3,5-Tris-(N-(DO3A-acetamido)-N-methyl-4-amino-2-methyl-phenyl)-[1,3,5]triazinane-2,4,6-trione (10)

The crude product of 9 (2.4 g) was dissolved in water and Gd(OAc)₃ (1.4 g, 4.2 mmol) was added under stirring. Vacuum (0.3 mbar) was then put on and the reaction was monitored continuously by LC/MS. When complete complexation was detected, the solvents were removed in vacuo. The crude product of 3.1 g was then purified by preparative HPLC (410 mg, 42% from 7)

### Example 5

### Dynamic Nuclear Polarisation of an Aqueous Solution Containing Caesium Pyruvate, a Trityl Radical as the DNP Agent and a Gd-chelate as a Paramagnetic Metal Ion

An aqueous solution containing the Cs-pyruvate and trityl radical as described in Example 3 was prepared. To this solution, 3 μl of a solution of the Gd-chelate of Example 4 in water was added. The final aqueous solution was about 14 mM in trityl radical and 0.7 mM in the Gd-chelate of Example 4 (2.1 mM with respect to Gd³⁺). From this solution 65 d were transferred to a probe cup and inserted in a DNP polariser. The frozen probe was polarised under DNP conditions at 1.2 K in a 3.35 T magnetic field under microwave irradiation (93.950 GHz). After 2 hours, the polarisation was stopped.

After the polarisation had been stopped, the frozen probe was dissolved in 7 ml water containing 100 mg/l EDTA and liquid state polarisation of the ¹³C nuclei in the hyperpolarised caesium pyruvate was determined by liquid state ¹³C-NMR at 400 MHz to be 24%.

### Example 6

### Dynamic Nuclear Polarisation of an Aqueous Solution Containing Caesium Pyruvate, a Trityl Radical as the DNP Agent and a Gd-chelate as a Paramagnetic Metal Ion and Ion Exchange of Caesium

DNP and dissolution was carried out as described in Example 5. After dissolution, the liquid containing the hyperpolarised caesium pyruvate was forced through a wetted, sodium charged ion exchange column which had been prepared in analogy to Example 1. The first 2 ml of the eluate was discharged. The total time of the ion exchange procedure was about 4-5 s.

Liquid state polarisation of the ¹³C nuclei in the hyperpolarised sodium pyruvate was determined by liquid state ¹³C-NMR at 400 MHz to be 17%.

### Example 7

### Dynamic Nuclear Polarisation of an Aqueous Solution Containing Caesium Bicarbonate and a Trityl Radical as the DNP Agent

A solution being 10 mM in trityl radical was prepared by dissolving tris-(8-carboxy-2,2,6,6-tetra (hydroxyethoxy)methylbenzo[1,2-d:4,5-d′]bis(1,3)-dithio-4-yl)methyl sodium salt which was prepared as described in Example 29 of WO-A-97/09633 in a solution of 21 mg caesium ¹³C-bicarbonate in 5 μl glycerol and 8 μl water. The solution was mixed to homogeneity by a combination of vortex, light heating and sonication, placed in a probe cup and inserted in a DNP polariser. The frozen probe was polarised under DNP conditions at 1.2 K in a 3.35 T magnetic field under microwave irradiation (93.890 GHz). After 3 hours, the polarisation was stopped.

The solid state polarisation of the ¹³C nuclei in the hyperpolarised caesium ¹³C-bicarbonate was determined by solid state ¹³C-NMR to be 70 (integral/mmol ¹³C).

### Example 8

### Dynamic Nuclear Polarisation of an Aqueous Solution Containing Caesium Bicarbonate, a Trityl Radical as the DNP Agent and a Gd-chelate as a Paramagnetic Metal Ion

A solution was prepared according to Example 7. To this solution was added the Gd-chelate of Example 4 resulting in a solution being 0.7 mM in Gd-chelate of Example 4 (2.1 mM with respect to Gd³⁺). The solution was mixed to homogeneity by a combination of vortex, light heating and sonication, placed in a probe cup and inserted in a DNP polariser. DNP was carried out as described in Example 7.

The solid state polarisation of the ¹³C nuclei in the hyperpolarised caesium ¹³C-bicarbonate was determined by solid state ¹³C-NMR to be 390 (integral/mmol ¹³C).

### Example 9

### Dynamic Nuclear Polarisation of an Aqueous Solution Containing Caesium Bicarbonate, a Trityl Radical as the DNP Agent and a Gd-chelate as a Paramagnetic Metal Ion

A solution being 12 mM in trityl radical was prepared by dissolving the trityl radical tris-(8-carboxy-2,2,6,6-tetra (hydroxyethoxy)methylbenzo[1,2-d:4,5-d′]bis(1,3)-dithiol-4-yl)methyl sodium salt which was prepared as described in Example 29 of WO-A-97/09633 in a solution of 0.205 mmol caesium ¹³C-bicarbonate in 12 μl glycerol and 16 μl water. The Gd-chelate of Example 4 was added to this solution resulting in a solution being 0.2 mM in the Gd-chelate (0.6 mM in Gd3+). The solution was mixed to homogeneity by a combination of vortex, light heating and sonication, placed in a probe cup and inserted in a DNP polariser. The frozen probe was polarised under DNP conditions at 1.2 K in a 3.35 T magnetic field under microwave irradiation (93.890 GHz). After 3 hours, the polarisation was stopped and the frozen solution was dissolved in an aqueous solution using a dissolution device according to WO-A-02/37132. A solution being 10 mM in hyperpolarised caesium ¹³C-bicarbonate was obtained.

Liquid state polarisation of the ¹³C nuclei in the hyperpolarised caesium bicarbonate was determined by liquid state ¹³C-NMR at 400 MHz to be 18%.

1-20. (canceled)

21. Method of producing a hyperpolarised carboxylate or sulphonate or mixtures thereof, the method comprising
a) preparing a solution comprising a carboxylate or a sulphonate or mixtures thereof wherein the carboxylate and/or sulphonate comprises an inorganic cation from the group consisting of NH₄⁺, K⁺, Rb⁺, Cs⁺, Ca²⁺, Sr²⁺ and Ba²⁺, a DNP agent and optionally a paramagnetic metal ion;
b) freezing the solution;
c) carrying out dynamic nuclear polarisation on the frozen solution to obtain a frozen solution comprising the hyperpolarised carboxylate or the hyperpolarised sulphonates or mixtures thereof; and
d) optionally liquefying the frozen solution obtained in step c).

22. Method according to claim 21 wherein the solution comprises a carboxylate, preferably an endogenous carboxylate and more preferably an endogenous carboxylate that plays a role in a metabolic process in the human or non-human animal body.

23. Method according to claim 22 wherein the carboxylate is a ¹³C enriched carboxylate.

24. Method according to claim 22 wherein the carboxylate is malate, acetate, fumarate, lactate, citrate, pyruvate, bicarbonate, malonate, carbonate, succinate, oxaloacetate, α-ketoglutarate, 2-oxobutanoate, 2-oxo-5-methylpentanoate, γ-carboxyglutamate, pyridine-2,3-dicarboxylate or isocitrate.

25. Method according to claim 21 wherein the inorganic cation is NH₄⁺, K⁺, Rb⁺ or Cs⁺, preferably K⁺, Rb⁺ or Cs⁺ and more preferably Rb⁺ or Cs⁺.

26. Method according to claim 21 wherein the solution is at least 5 molar in carboxylate or in sulphonate or in mixtures thereof.

27. Method according to claim 21 wherein the DNP agent is a stable oxygen-based, sulphur-based or carbon-based trityl radical.

28. Method according to claim 21 wherein the solution comprises a paramagnetic metal ion, preferably a paramagnetic metal ion of a lanthanide metal of atomic numbers 58-70 or of a transition metal of atomic numbers 21-29, 42 or 44.

29. Composition comprising a carboxylate or a sulphonate or mixtures thereof wherein said carboxylate or sulphonate comprises an inorganic cation from the group consisting of NH₄⁺, K⁺, Rb⁺, Cs⁺, Ca²⁺, Sr²⁺ and Ba²⁺, a DNP agent and optionally a paramagnetic metal ion.

30. Composition comprising a hyperpolarised carboxylate or hyperpolarised sulphonates or mixtures thereof wherein said hyperpolarised carboxylate or hyperpolarised sulphonates comprises an inorganic cation from the group consisting of NH₄⁺, K⁺, Rb⁺, Cs⁺, Ca²⁺, Sr²⁺ and Ba²⁺.

31. Composition according to claim 29 wherein the composition comprises a carboxylate, preferably an endogenous carboxylate and more preferably an endogenous carboxylate that plays a role in a metabolic process in the human or non-human animal body.

32. Composition according to claim 29 wherein the carboxylate is a ¹³C enriched carboxylate.

33. Composition according to claim 29 wherein the carboxylate is a carboxylate from the group consisting of malate, acetate, fumarate, lactate, citrate, pyruvate, bicarbonate, malonate, carbonate, succinate, oxaloacetate, α-ketoglutarate, 2-oxobutanoate, 2-oxo-5-methylpentanoate, γ-carboxy-glutamate, pyridine-2,3-dicarboxylate and isocitrate.

34. Composition according to claim 29 wherein the inorganic cation is NH₄⁺, K⁺, Rb⁺ or Cs⁺, preferably K⁺, Rb⁺ or Cs⁺ and more preferably Rb⁺ or Cs⁺.

35. Composition according to claim 30 being obtained by dynamic nuclear polarisation.
